(19) United States
(12) Patent Application Publication
(10) Pub. No.: US 2014/0350433 A1
(43) Pub. Date: Nov. 27, 2014

(54) PRESSURE MEASURING DEVICE AND FIXING DEVICE WITH PRESSURE MEASURING FUNCTION FOR AFFECTED PART

(71) Applicants: Fuh-Yu Chang, Jhubei City (TW); Chieh-Hsiao Chen, Taichung City (TW)

(72) Inventors: Fuh-Yu Chang, Jhubei City (TW); Chieh-Hsiao Chen, Taichung City (TW)

(21) Appl. No.: 14/285,485

(22) Filed: May 22, 2014

(30) Foreign Application Priority Data
May 23, 2013 (TW) 102118172

(51) Int. Cl. A61B 5/03; A61F 5/01

(52) U.S. Cl. CPC A61B 5/03; A61F 5/0102; A61F 2005/0188
USPC 600/561

(57) ABSTRACT

A pressure measuring device for measuring the internal pressure of the affected part under the fixing member includes a plurality of flexible pressure meters (or called as manometers) and a processor connected to each of the flexible pressure meters. The flexible pressure meters are configured at the inner surface of the fixing member and pasted on the affected part to measure the surface pressure of the affected part, and generate measuring signals according to the measured pressure. The processor is capable of receiving the measuring signals and displaying the surface pressure of the affected part according to the measuring signals, furthermore, by analyzing the measuring signals, the internal pressure corresponded to each of the compartments of the affected part can be found. Accordingly, the pressure measuring device assists to judge if compartment syndrome occurs in the affected part.

### PRIORITY CLAIM

This application claims the benefit of the filing date of Taiwan Patent Application No. 102118172, filed May 23, 2013, entitled “PRESSURE MEASURING DEVICE AND FIXING DEVICE WITH PRESSURE MEASURING FUNCTION FOR AFFECTED PART,” and the contents of which is hereby incorporated by reference in its entirety.

### FIELD OF THE INVENTION

The present invention discloses a pressure measuring device and a fixing device with pressure measuring function for affected part. More specifically, the present invention discloses a pressure measuring device and a fixing device adapted to measure the pressure of affected parts of a patient by calculating the pressure distribution of the affected limbs so as to be the diagnosis of compartment syndrome.

### BACKGROUND OF THE INVENTION

The limbs of human has a base of the skeletal system, which the bones are encased by nerve, blood vessels and muscles having fascia, subcutaneous tissue and skin formed thereon. These tissues are formed around the limbs and form a plurality of chamber liked compartments; the compartment is flexible and can be expended and compressed. FIG. 1 illustrates a schematic sectional view of a calf of a human being. As shown in the FIG. 1, the calf 1 comprise at least one tibia 100, a fibula 102, a front side compartment 12, a linear side compartment 14, a back side deep layer compartment 16 and a back side surface compartment 18, wherein, each of the said compartments comprises a group of muscles respectively.

While the limb is seriously injured, apart from the injury directly caused by an external force applied to the limb, follow-up damage caused by the blood or tissue fluid generated by the injured tissue is also possible, for example, compartment syndrome. Compartment syndrome is one of the said follow-up damage may caused; it is an emergency situation of orthopedic. While the limb compartments are injured and excess liquid (such as blood or tissue fluid) is accumulated therein, the pressure in the tissue may raise and the large pressure thereof may cause the severe swelling of tissue and may block the blood circulation of the muscle and nerve tissue, resulting in hypoxia and necrosis of tissue.

Compartment syndrome can commonly be found in some of the cases that bone closed fracture with comminuted bone fracture, usually, the tissue bleed massively and the blood will flow into the compartments, resulting in the rise of the pressure inside the compartment. Under normal circumstances, the venous pressure and the pressure of the compartments is close to zero, however, while the tissue is subjected to severe trauma, the venous pressure increased rapidly, once the deviations of the compartment pressure and diastolic blood pressure become less than 20 to 40 mm Hg, the blood of the venous shall not able to reflux, resulting in tissue swelling, further increasing the pressure thereof, and may lead to necrosis of the muscle tissue and may require cut limbs or the patient may face a life-threatening situation.

In addition to the trauma type of compartment syndrome as previously described, no matter crushing, burning or even plaster or other possible situations may also cause compartment syndrome.

Plaster is widely used to fix the affected parts when the patient is facing bone fracture or trauma situation. However, if the affected part keep swelling after the plaster is encased thereat, the linear side of the plaster may restrict the swelling thereof, causing the accumulation of the pressure in the compartment and aggravating the syndrome. Further, since the affected part is covered by plaster, neither the patient nor the medical personnel may know the situation of the affected part, which the pain caused by compartment syndrome can easily confused with the pain caused by bone fracture hindering the diagnosis of compartment syndrome.

### SUMMARY OF THE INVENTION

Accordingly, one of the aspect of the present invention is to provide a pressure measuring device that capable of measuring the internal pressure of the affected part encased in the fixing member so as to solve the problem of the prior art.

In one embodiment, the present invention discloses a pressure measuring device which comprises a plurality of flexible manometers and a processor, wherein, each of the flexible manometers may be disposed at an inner surface of a fixing member, the said fixing member may comprises a plaster or any other fixing cover formed of plastic or clothes based material. Each of the plurality of flexible manometers is adapted to be pasted on the affected part and measure the surface pressure of the affected part respectively so as to generate a measuring signal in accordance with the surface pressure measured thereby. Accordingly, the patient or the medical personnel may know the pressure of the affected part inside the plaster so as to assist diagnose of compartment syndrome. One of the main features of the present invention is to obtain the pressure variation of the compartments by analyzing the variations of the pressure or tension of the surface of the skin.

The another aspect of the present invention is to provide an affected part fixing device for measuring internal pressure of the affected part so as to solve the problem of the prior art. In another embodiment, the affected part fixing device of the present invention may measure the surface pressure of the affected part, the device may comprises a fixing member encased and fixed on an affected part, a plurality of flexible manometers and a processor. The said fixing member may be a plaster or any other fixing cover formed of plastic or clothes based material. Each of the flexible manometers is disposed on/at the inner surface of the fixing member and to be pasted on the affected part, the plurality of flexible manometers can measure the pressure of the affected part respectively so as to generate a measuring signal accordingly. The processor may be connected with the plurality of flexible manometers so as to receive the measuring signals, the processor is for generating and outputting a surface pressure of the affected part in accordance with the measuring signal.

### BRIEF DESCRIPTION OF THE DRAWINGS

The advantage and spirit of the invention may be understood by the following recitations together with the appended drawings.

FIG. 1 illustrates a schematic sectional view of a calf.

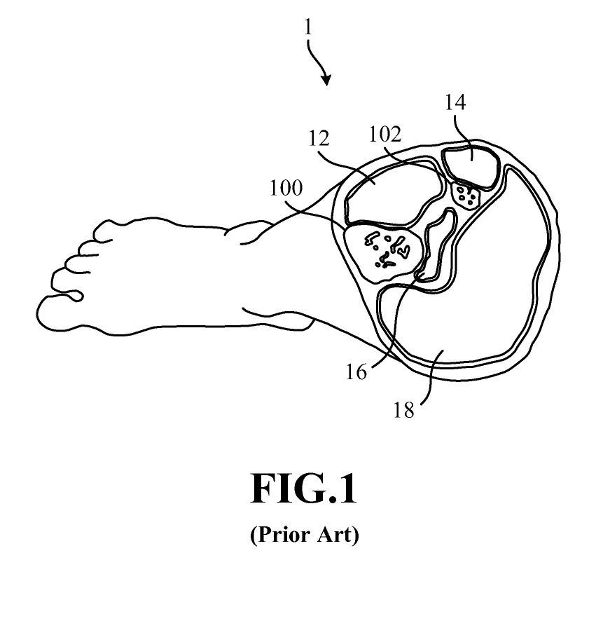

FIG. 2 illustrates a schematic figure of a pressure measuring device.

FIG. 3 illustrates a schematic sectional view of another embodiment of the pressure measuring device.

### DETAILED DESCRIPTION

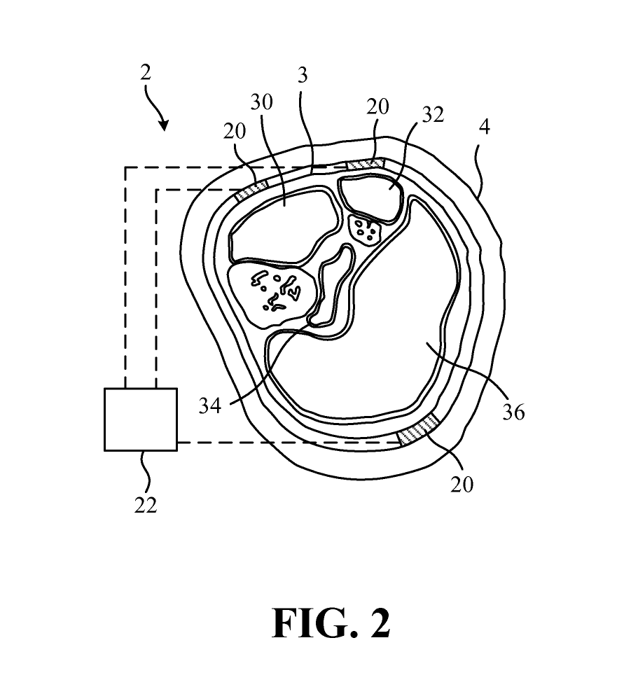

FIG. 2 illustrates a schematic figure of a pressure measuring device 2. As shown in FIG. 2, the fixing member 4 encases an affected part 3 so as to provide fixing and supporting effect thereto. The said fixing member 4 may refer to a plaster or any other fixing cover formed of plastic or clothes based material. In the present embodiment, the fixing member 4 refers to a plaster. The affected part 3 may be a limb of a human being or any other animals. When the limb is serious injured, such as bone fracture, the bone fails to provide support force, accordingly, a plaster 4 is usually encased onto the affected part 3 so as to provide the function of fixing and supporting so as to accurate the recuperation of the affected part 3. In present embodiment, the affected part 3 is referred to a calf of human being, however, in actual practice, the affected part 3 is not limited to calf of human being and it can also be other bone of limb apart from calf that may need the plaster to help the fixation thereof.

The pressure measuring device 2 comprises a plurality of flexible manometers 20, which is disposed on or at the inner side or inner surface of the plaster 4 close to the affected part 3. More precisely, the flexible is disposed at the predetermined position corresponding and close to the affected part 3. Generally, plaster 4 contacts with the skin (or called as the outermost layer) of the body, and the flexible manometer 20 can be pasted onto the skin of the affected part 3. Furthermore, the flexible manometer 20 can be formed of a soft based material, for example, high polymer phase-change material (PCM) material or high polymer with carbon nano-fibers material. Considering the flexible manometer 20 is soft, and may be bendable, therefore, it can be pasted closely to the silhouette of skin of the affected part 3. Furthermore, in actual practice, hydrophilicity treatment can be processed to the surface of flexible manometer 20 so as to fix the flexible manometer 20 tightly with the skin without causing the effort of skin.

The flexible manometer 20 is adapted to measure the surface pressure of the affected part 3 and generate a surface pressure measuring signal accordingly. The pressure measuring device 2 further comprises a processor 22, which is connected with the plurality of flexible manometers 20 so as to receive the measuring signal generated by the plurality of flexible manometers 20. By computing the measuring signal, the processor 22 can get the surface pressures of where the manometers 20 are disposed respectively. Furthermore, an internal pressure of the affected part and the variation thereof can also be found by the further computing the measuring signals. In actual practice, the processor 22 may comprise or connects with a monitor, for displaying the said pressures. Moreover, the pressure or the variation thereof may be displayed by value or figure, for example, the monitor may display a plurality of value corresponding to the surface pressure detected by the flexible manometers 20, or display a pressure distribution chart corresponding to the affected part 3.

As shown in FIG. 2, the affected part 3 comprises four compartments of front side compartment 30, linear side compartment 32, back side deep layer compartment 34 and back side surface compartment 36. In this embodiment, the pressure measuring device 2 comprises three flexible manometers 20, which each of the flexible manometers is disposed on the predetermined position corresponding to the front side compartment 30, linear side compartment 32 and back side surface compartment 36. Each of the flexible manometers 20 can measure the pressure of the corresponding compartment and send the measuring signal to the processor 22 respectively, allowing the processor 22 to display the surface or internal pressures corresponding to each of the compartments of front side compartment 30, linear side compartment 32 and back side surface compartment 36. In another way, the processor 22 can also simulates the internal pressure of the compartments or the whole limb, according to the signals generated by the flexible manometers 20, and displays it in characters or figure. Accordingly, the processor 22 can also display the pressure of the front side compartment 30, linear side compartment 32, back side surface compartment 36 and back side deep layer compartment 34. While the affected part 3 has the situation of compartment syndrome, the inner pressure of the compartment alone or the whole affected part 3 will rise abnormally, by the pressure measuring device 2, the change can be detected and shown. Accordingly, the medical personnel may diagnose if there is compartment syndrome according to the pressure abnormal rise displayed by processor 22 and determined if the plaster 4 should be removed so as to avoid further injury caused to affected part 3.

It should be noticed that, the said flexible manometers 20 are disposed on various positions corresponding to the compartments, however, in actual practice, the positions of flexible manometers are disposed and the quality thereof may be adjusted by the situation of the patient or the judgment of the medical personnel and it is not limited by the present invention. For example, if the medical personnel wants to know if a certain compartment of the affected part will have the problem of compartment syndrome, more flexible manometer may be disposed at the position corresponding to the certain compartment so as to understand the pressure situation of the certain compartment more preciously. In another way, if one compartment is relatively large, it may require more flexible manometers to measure the pressure of the whole compartment.

In the present embodiment, processor 22 is disposed outside the plaster 4 and the affected part 3, for example, the processor 22 may be disposed in an inspection instrument in the consulting room of hospital. And the patient can check the pressure of the affected part 3 in the hospital by the inspection instrument. In the present embodiment, the flexible manometer 20 is connected with processor 22 via a wireless means. In another word, the flexible manometer 20 can send the measuring signal to the processor 22 via the wireless transmitting means so as to process thereto and display the pressure of the affected part 3. However, the processor 22 is not limited to be disposed outside of the plaster 4 and affected part 3, and the connection between the flexible manometer 20 and the processor 22 can also be done by wire.

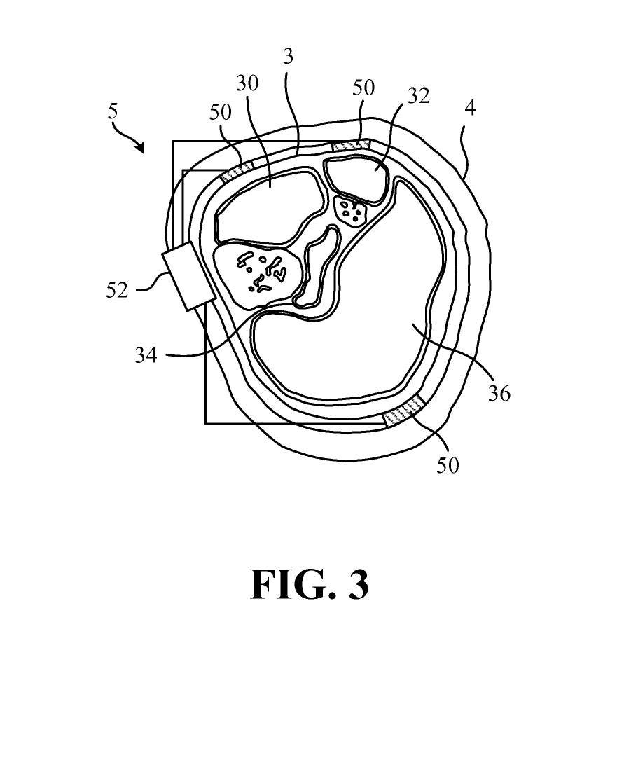

FIG. 3 illustrates a schematic sectional view of another embodiment of the pressure measuring device 5. As shown in FIG. 3, one of the major differences between the present embodiment and the previous embodiment is that, the processor 52 of the pressure measuring device 5 is disposed on/at the plaster 4 and connected with flexible manometer 50 by wire. In another word, the flexible manometer 50 can send the measuring signal measured thereby to the processor for analyzing and computing so as to output or to display the pressure of the affected part 3. The processor 52 may comprise a display module or monitor so as to display the pressure of a compartment or the whole affected part 3.

Furthermore, since the processor 52 is disposed on/at the plaster 4, the patient or the medical personnel may know the pressure of the affected part 3 so as to achieve the goal of real time monitoring. It should be noticed that, the connecting cable depicted in the FIG. 3 among the flexible manometers 50 and processor 52 actually means the items are connected in wire, but should not be understood as the limitation of the relative position between the connecting cable and plaster 4. In actual practice, the connecting cable between the flexible manometer 50 and processor 52 can be embedded in the plaster 4 for providing a nice cosmetic so as to avoid patient or anyone to cut off the connecting cable accidently.

Plaster is widely used to fix the affected parts when the patient is facing bone fracture or trauma situation. However, if the affected part keeps swelling after the plaster is encased thereat, the linear side of the plaster may restrict the swelling thereof for causing the accumulation of the pressure in the compartment and aggravating the syndrome. The present invention provides an affected part fixing device, which provides affected part supporting function and also assists to judge if the affected part has the situation of compartment syndrome. In another embodiment, the present invention of affected part fix device comprises a plaster, a flexible manometer and a processor as shown in FIG. 2, and the details thereof shall be herein described.

In the present embodiment, affected part fixing device may fix the affected part 3. The affected part fixing device may comprise a plaster 4, a flexible manometer 20 and a processor 22. The plaster 4 is utilized to encase and fix the affected part 3 so as to provide a supporting force thereto. The flexible manometer 20 is disposed on/at the inner surface of the plaster 4 and also pasted on the affected part 3. The flexible manometer 20 may measure the surface pressure of the affected part 3 so as to generate a corresponding measuring signal. The processor 22 may be connected to the plurality of flexible manometers 20 so as to receive the measuring signal thereby. Then the measuring signal will be processed so as to have the surface pressure and internal pressure of the affected part by collecting the surface pressure of the plurality of flexible manometers 20 and display those surface pressures or the corresponding limb internal pressure. In another word, the affected part fixing device is a fixing device for measuring the pressure of the affected part.

Likewise, processor of the affected part fixing device of the present invention may be disposed on/at the plaster or outside of the plaster and connect with the flexible manometers by wire or wireless means. As shown in FIG. 2 and FIG. 3, the pressure of the affected part generated by the processor of the affected part fixing device will assist patient or medical personnel to be the evidence of compartment syndrome. If the processor shows that the pressure of affected part excesses a certain level, for example, if the deviations of the compartment pressure and diastolic blood pressure (internal pressure obtained by the processor 22) are among 30 to 40 mmHg, the medical personnel may know there will be the situation of compartment syndrome and could remove the plaster so as to treat therewith.

In summary, the pressure measuring device and affected part fixing device of the present invention comprise at least one flexible manometer disposed on or at the inner surface of the plaster, the flexible manometer may also be pasted onto the affected part, and connected with the processor. The flexible manometer may measure the pressure of affected part and generate a measuring signal accordingly. The processor may receive measuring signal for the computing so as to simulate or obtain the internal pressure of the compartment of the affected part or the whole affected part. In comparison to the prior art, the present invention may detect the internal pressure of the affected part inside the plaster in real time, so as to help patient and medical personnel determine if the affected part has the situation of compartment syndrome, avoid the further serious injury caused by plaster and allow the medical personnel to treat with the compartment syndrome on time.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

1. A pressure measuring device, adapted to obtain an internal pressure of an affected part encased in a fixing member, the pressure measuring device comprising:
a plurality of flexible manometers, disposed at an inner surface of the fixing member, each of the plurality of flexible manometers adapted to be pasted on the affected part and measuring a surface pressure of the affected part so as to generate a measuring signal in accordance with the surface pressure measured thereby respectively; and
a processor, connected to the plurality of flexible manometers so as to receive the measuring signals, for generating and outputting the surface pressures, the internal pressures of the affected part or the variation thereof by computing the measuring signals.

2. The pressure measuring device of the claim 1, wherein the plurality of flexible manometers are disposed on or at the inner surface of the fixing member and pasted onto a plurality of predetermined positions on the affected part, the plurality of predetermined positions are corresponded to the position of a plurality of compartments in the affected part.

3. The pressure measuring device of the claim 2, wherein the processor simulates and outputs the internal pressure of the plurality of compartments in accordance with the measuring signals generated by the plurality of manometers.

4. The pressure measuring device of the claim 1, wherein the processor is connected with the plurality of flexible manometers by wireless means.

5. The pressure measuring device of the claim 1, wherein the processor is disposed on the fixing member and connected with the plurality of flexible manometers by wire.

6. A fixing device for measuring an internal pressure of an affected part, comprising:
a fixing member, encasing and fixing the affected part;
a plurality of flexible manometers, disposed at an inner surface of the fixing member and pasted on the affected part, each of the plurality of flexible manometers measuring a surface pressure of the affected part so as to generate a measuring signal in accordance with the pressure measured thereby respectively; and
a processor, connected to the plurality of flexible manometers so as to receive the measuring signals, the processor generating and outputting a plurality of corresponding internal pressures of the affected part in accordance with the measuring signals.

7. The fixing device of claim 6, wherein the plurality of flexible manometers are pasted onto a plurality of predetermined positions of the affected part, each of the plurality of predetermined positions is corresponded to a compartment of the affected part.

8. The fixing device of claim 7, wherein the processor simulates and outputs the plurality of internal pressure of the plurality of compartments in accordance with the measuring signals generated by the plurality of manometers.

9. The fixing device of claim 6, wherein the processor are connected with the plurality of flexible manometers by wireless means.

10. The fixing device of claim 6, wherein the processor is disposed on the fixing member and connected with the plurality of flexible manometers by wire.
